(19) United States
(12) Patent Application Publication
(10) Pub. No.: US 2026/0157649 A1
(43) Pub. Date: Jun. 11, 2026

(54) VITAL INFORMATION ACQUISITION APPARATUS AND METHOD

(71) Applicant: MARI CO., LTD., Kyoto (JP)

(72) Inventors: Takuya SAKAMOTO, Kyoto (JP); Toru SATO, Kyoto (JP); Shigeaki OKUMURA, Kyoto (JP); Hirofumi TAKI, Kyoto (JP); Itsuki IWATA, Kyoto (JP); Takato KODA, Kyoto (JP)

(21) Appl. No.: 18/706,894

(22) PCT Filed: Nov. 7, 2022
(86) PCT No.: PCT/IB2022/000663

§ 371 Date: May 2, 2024

Related U.S. Application Data
(60) Provisional application No. 63/275,949, filed on Nov. 5, 2021.
(60) Provisional application No. 63/299,958, filed on Jan. 15, 2022.
(60) Provisional application No. 63/414,559, filed on Oct. 9, 2022.

(51) Int. Cl. A61B 5/0507; A61B 5/00; A61B 5/02; A61B 5/0205; A61B 5/021; A61B 5/024; A61B 5/08; A61B 7/00

(52) U.S. Cl. CPC A61B 5/0507; A61B 5/02028; A61B 5/0205; A61B 5/021; A61B 5/024; A61B 5/0816; A61B 5/4812; A61B 5/4818; A61B 5/7239; A61B 5/725; A61B 5/7257; A61B 5/7271; A61B 7/003

(57) ABSTRACT

The present invention aims at a vital information acquisition apparatus, comprising: a microwave radar system which includes at least one transmitting antenna and at least one receiving antenna and is configured to transmit a plurality of microwaves to a subject and receive a plurality of microwaves reflected by the subject; and a controller comprising circuitry configured to convert a plurality of received microwaves to a plurality of radar signals, store the radar signals, calculate the phase signals of the radar signals, and calculate the first- or higher-order derivatives of each phase signal, and estimate heartbeat intervals and/or respiratory intervals from the phase signals and/or their derivatives using one of periodicity detection techniques; the periodicity finding techniques include autocorrelation, frequency analysis using Fourier transform, and zero-crossing detection algorithm. Another aspect of the present invention is a vital information acquisition method that stores the radar signals, calculates the phase signals of the radar signals, and calculates the first- or higher-order derivatives of each phase signal, and estimates heartbeat intervals and/or respiratory intervals from the phase signals and/or their derivatives using one of periodicity detection techniques; the periodicity finding techniques include autocorrelation, frequency analysis using Fourier transform, and zero-crossing detection algorithm.

### CROSS-REFERENCE TO RELATED APPLICATIONS

The present application is based upon and claims the benefits of priority to U.S. Provisional Application No. 63/275,949, filed Nov. 5, 2021, U.S. Provisional Application No. 63/299,958, filed Jan. 15, 2022, and U.S. Provisional Application No. 63/414,559, filed Oct. 9, 2022. The entire contents of all of the above applications are incorporated herein by reference.

### TECHNICAL FIELD

The present invention is directed to a vital information acquisition apparatus and method that estimate respiratory intervals, heartbeat intervals, and positions of a subject using a millimeter-wave radar.

### BACKGROUND ART

Vital information monitoring is very important in order to provide appropriate healthcare services to patients (PL 1, NPL 1). Recently, several millimeter-wave radar techniques (PL 2, NPL 2, NPL 3) have been reported for the acquisition of vital information including heart rate and respiratory interval.

### CITATION LIST PATENT LITERATURE

PL 1 Katsuya Nakagawa, et. al. Vital information measuring device, managing device, and vital information communication system, EP1887488A1.
PL 2 Milan Savic, et. al., MM-wave radar vital signs detection apparatus and method of operation, WO2015/174879A1.

### CITATION LIST NON PATENT LITERATURE

NPL 1 Sandy Rolfe, The importance of respiratory rate monitoring, British Journal of Nursing, 2019.
NPL 2 Zhicheng Yang, et. al., Monitoring vital signs using millimeter wave, MobiHoc' 16, 2016.
NPL 3 Takuya Sakamoto, Recent progress in millimeter-wave radar signal processing, 12^{th} Global Symposium on Millimeter Waves, 2019.

### SUMMARY OF THE INVENTION

Vital information monitoring is very important in order to provide appropriate healthcare services to patients. For the improvement of healthcare service quality, non-contact and robust vital information monitoring techniques are strongly desired.

To solve the above-mentioned problem, the present invention aims at a vital information acquisition apparatus, comprising: a microwave radar system which includes at least one transmitting antenna and at least one receiving antenna and is configured to transmit a plurality of microwaves to a subject and receive a plurality of microwaves reflected by the subject; and a controller comprising circuitry configured to convert a plurality of received microwaves to a plurality of radar signals, store the radar signals, calculate the phase signals of the radar signals, and calculate the first- or higher-order derivatives of each phase signal, and estimate heartbeat intervals and/or respiratory intervals from the phase signals and/or their derivatives using one of periodicity detection techniques; the periodicity finding techniques include autocorrelation, frequency analysis using Fourier transform, and zero-crossing detection algorithm.

Another aspect of the present invention is a vital information acquisition method that stores the radar signals, calculates the phase signals of the radar signals, and calculates the first- or higher-order derivatives of each phase signal, and estimates heartbeat intervals and/or respiratory intervals from the phase signals and/or their derivatives using one of periodicity detection techniques; the periodicity finding techniques include autocorrelation, frequency analysis using Fourier transform, and zero-crossing detection algorithm.

### BRIEF DESCRIPTION OF DRAWINGS

A more complete appreciation of the invention and many of the attendant advantages thereof will be readily obtained as the same becomes better understood by reference to the following detailed description when considered in connection with the accompanying drawings, wherein:

FIG. 1 is a schematic diagram of a vital information acquisition apparatus that transmits ultra-wideband millimeter-waves to a subject and estimates vital information of the subject from the phase signals and/or their derivatives using a periodicity finding technique.

FIG. 2 is a schematic diagram of a vital information acquisition apparatus that applies one of multi-valued filters to the first-, second- and third-order derivatives of the phase signals.

FIG. 3 is a schematic diagram of a vital information acquisition apparatus that selects appropriate positions or distances for the estimation of heartbeat intervals and respiratory intervals, where the appropriate position or distance for the estimation of heartbeat intervals and the appropriate position or distance for the estimation of respiratory intervals do not have to be the same.

FIG. 4 is a schematic diagram of a vital information acquisition apparatus that applies band-path filter to the phase signals of the radar signals, the radar signals, and/or the derivatives of the phase signals of the radar signals.

FIG. 5 is a schematic diagram of a vital information acquisition apparatus that transmits ultra-wideband millimeter-waves to a subject and estimates vital information of the subject by calculating phase signal from the radar signals at plural positions or distance.

FIG. 6 is a schematic diagram of acquisition apparatus that calculates the phase signal using one of techniques based on principal component analysis.

FIG. 7 is a schematic diagram of acquisition apparatus that employs one of techniques based on principal component analysis and applies band-pass filter to radar signals and phase signals.

FIG. 8 a schematic diagram of acquisition apparatus that employs one of techniques based on principal component analysis, applies band-pass filter or high-pass filter to radar signals, and estimates vital information of the subject from the phase signals and/or their derivatives after band-pass filter application.

FIG. 9 is a schematic diagram of a vital information acquisition apparatus that transmits ultra-wideband millimeter-waves to a subject, receives a plurality of sound including snoring sounds, and estimates vital information of the subject by calculating phase signal of the radar signals at plural positions or distance and periodicity of the existence time distribution of the sound impact comprising sound pressure.

### DESCRIPTION OF EMBODIMENTS

The embodiments will now be described with reference to the accompanying drawings, wherein like reference numerals designate corresponding or identical elements throughout the various drawings.

Vital information acquisition apparatus of the present invention according to an embodiment transmits ultra-wideband millimeter-waves and estimates heartbeat intervals and respiratory intervals of the subject from the phase signals of the radar signals and their derivatives using a periodicity detection technique. A vital information acquisition apparatus of the present invention according to an embodiment is comprising a microwave radar system which includes at least one transmitting antenna and at least one receiving antenna and is configured to transmit a plurality of microwaves to a subject and receive a plurality of microwaves reflected by the subject; and a controller comprising circuitry configured to convert a plurality of received microwaves to a plurality of radar signals, store the radar signals, calculate the phase signals of the radar signals, and calculate the first- or higher-order derivatives of each phase signal, and estimate heartbeat intervals and/or respiratory intervals from the phase signals and/or their derivatives using one of periodicity detection techniques: the periodicity finding techniques include autocorrelation, frequency analysis using Fourier transform, and zero-crossing detection algorithm.

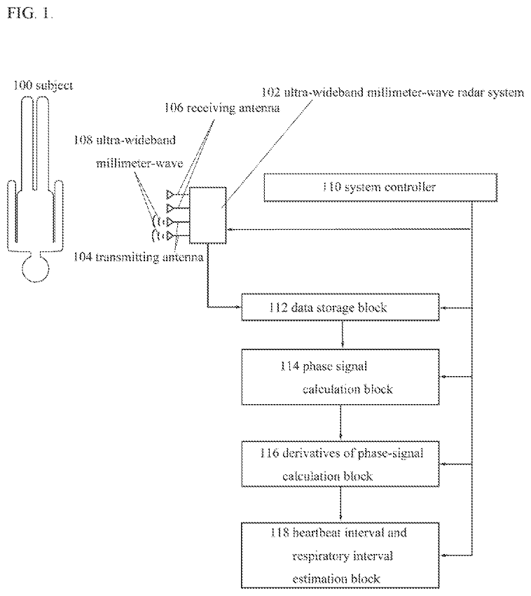

A vital information acquisition apparatus employing an embodiment of the present invention can employ an ultra-wideband millimeter-wave radar system. FIG. 1 shows a schematic diagram of a vital information acquisition apparatus employing an embodiment of the present invention. An ultra-wideband millimeter-wave radar system includes at least one transmitting antenna 104 and at least one receiving antenna 106. A plurality of ultra-wideband millimeter-waves 108 are transmitted to a subject 100. Transmitted ultra-wideband millimeter-waves can be modulated using one of pulse compression techniques, e.g. m-sequence. Transmitted ultra-wideband millimeter-waves 108 are reflected at the body surface of the subject 100. A plurality of ultra-wideband millimeter-waves reflected by the subject are received by receiving antennas 106. A system controller 110 comprising circuitry is configured to convert a plurality of received ultra-wideband millimeter-waves to a plurality of radar signals, store the radar signals 112, calculate the phase signals of the radar signals 114, and calculate the first- or higher-order derivatives of each phase signal 116, and estimate heartbeat intervals and/or respiratory intervals from the phase signals and/or their derivatives using one of periodicity detection techniques 118. The periodicity finding techniques include autocorrelation, frequency analysis using Fourier transform, and zero-crossing detection algorithm.

A vital information acquisition apparatus employing an embodiment of the present invention may calculate the first-, second- and third-order derivatives of each phase signal. The controller comprising circuitry is configured to calculate the first-, second- and third-order derivatives of each phase signal, and estimate heartbeat intervals and/or respiratory intervals from the phase signals and their first-, second- and third-order derivatives using one of periodicity detection techniques.

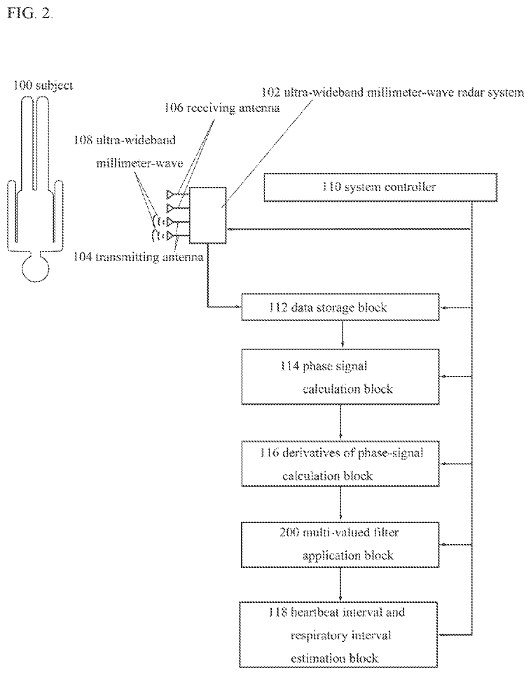

A vital information acquisition apparatus employing an embodiment of the present invention according may apply one of multi-valued filters to the first-, second- and third-order derivatives of the phase signals. FIG. 2 shows a schematic diagram of a vital information acquisition apparatus employing an embodiment of the present invention. An ultra-wideband millimeter-wave radar system includes at least one transmitting antenna 104 and at least one receiving antenna 106. A plurality of ultra-wideband millimeter-waves 108 are transmitted to a subject 100. Transmitted ultra-wideband millimeter-waves can be modulated using one of pulse compression techniques, e.g. m-sequence. Transmitted ultra-wideband millimeter-waves 108 are reflected at the body surface of the subject 100. A plurality of ultra-wideband millimeter-waves reflected by the subject are received by receiving antennas 106. A system controller 110 comprising circuitry is configured to convert a plurality of received ultra-wideband millimeter-waves to a plurality of radar signals, store the radar signals 112, calculate the phase signals of the radar signals 114, and calculate the first- or higher-order derivatives of each phase signal 116, apply one of multi-valued filters to the first-, second- and third-order derivatives of the phase signals 200, and estimate heartbeat intervals and/or respiratory intervals from the phase signals and/or their derivatives using one of periodicity detection techniques 118. The multi-valued filters include binarization filter. The periodicity finding techniques include autocorrelation, frequency analysis using Fourier transform, and zero-crossing detection algorithm.

A vital information acquisition apparatus employing an embodiment of the present invention may select appropriate position or distance from the radar signals and/or their phase signals in order to estimate heartbeat intervals and respiratory intervals. The controller comprising circuitry is further configured to select appropriate position or distance from the region of interest in order to estimate heartbeat intervals and respiratory intervals, and heartbeat intervals and respiratory intervals from the phase signals and/or their derivatives of the appropriate position or distance using one of periodicity detection techniques. A vital information acquisition apparatus employing an embodiment of the present invention may determine the position of the subject from the selected appropriate position or distance for the estimation of heartbeat intervals and/or respiratory intervals.

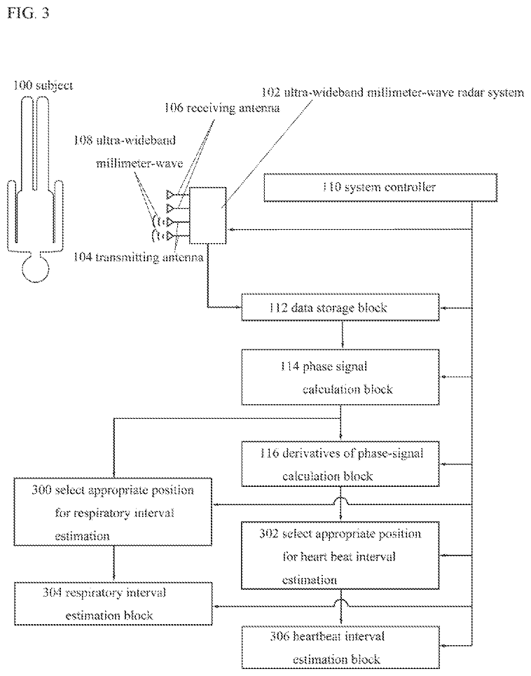

A vital information acquisition apparatus employing an embodiment of the present invention may select appropriate positions or distances for the estimation of heartbeat intervals and respiratory intervals, where the appropriate position or distance for the estimation of heartbeat intervals and the appropriate position or distance for the estimation of respiratory intervals do not have to be the same. FIG. 3 shows a schematic diagram of a vital information acquisition apparatus employing an embodiment of the present invention. An ultra-wideband millimeter-wave radar system includes at least one transmitting antenna 104 and at least one receiving antenna 106. A plurality of ultra-wideband millimeter-waves 108 are transmitted to a subject 100. Transmitted ultra-wideband millimeter-waves can be modulated using one of pulse compression techniques, e.g. m-sequence. Transmitted ultra-wideband millimeter-waves 108 are reflected at the body surface of the subject 100. A plurality of ultra-wideband millimeter-waves reflected by the subject are received by receiving antennas 106. A system controller 110 comprising circuitry is configured to convert a plurality of received ultra-wideband millimeter-waves to a plurality of radar signals, store the radar signals 112, calculate the phase signals of the radar signals 114, select appropriate position or distance from the region of interest for the estimation of respiratory intervals 300, calculate the first- or higher-order derivatives of each phase signal 116, apply one of multi-valued filters to the first-, second- and third-order derivatives of the phase signals 200, select appropriate position or distance from the region of interest for the estimation of heartbeat intervals 302, and estimate heartbeat intervals 306 and/or respiratory intervals 304 from the phase signals and/or their derivatives of the appropriate positions or distances using one of periodicity detection techniques, where the appropriate position or distance for the estimation of heartbeat intervals and the appropriate position or distance for the estimation of respiratory intervals do not have to be the same. The selection of appropriate position for respiratory interval estimation may follow the calculation of the derivatives of phase signals.

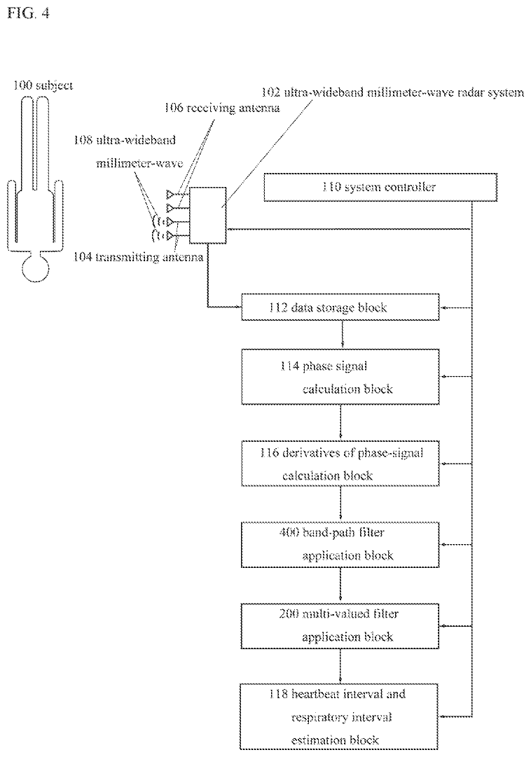

A vital information acquisition apparatus employing an embodiment of the present invention may apply band-path filter to the phase signals of the radar signals, the radar signals, and/or the derivatives of the phase signals of the radar signals. FIG. 4 shows a schematic diagram of a vital information acquisition apparatus employing an embodiment of the present invention. An ultra-wideband millimeter-wave radar system includes at least one transmitting antenna 104 and at least one receiving antenna 106. A plurality of ultra-wideband millimeter-waves 108 are transmitted to a subject 100. Transmitted ultra-wideband millimeter-waves can be modulated using one of pulse compression techniques. e.g. m-sequence. Transmitted ultra-wideband millimeter-waves 108 are reflected at the body surface of the subject 100. A plurality of ultra-wideband millimeter-waves reflected by the subject are received by receiving antennas 106. A system controller 110 comprising circuitry is configured to convert a plurality of received ultra-wideband millimeter-waves to a plurality of radar signals, store the radar signals 112, calculate the phase signals of the radar signals 114, and calculate the first- or higher-order derivatives of each phase signal 116, apply band-path filter to the phase signals of the radar signals and/or the derivatives of the phase signals of the radar signals 400, apply one of multi-valued filters to the first-, second- and third-order derivatives of the phase signals after band-path filter application 200, and estimate heartbeat intervals and/or respiratory intervals from the phase signals after band-path application and/or their derivatives using one of periodicity detection techniques 118. The multi-valued filters include binarization filter. The periodicity finding techniques include autocorrelation, frequency analysis using Fourier transform, and zero-crossing detection algorithm.

A vital information acquisition apparatus employing an embodiment of the present invention may calculate tentative heartbeat intervals and/or respiratory intervals among plural positions or distances, select the position or distance with the highest number of detected heartbeat intervals or respiratory intervals, and employ the detected heartbeat intervals or respiratory intervals of the selected position or distance as the estimated heartbeat intervals or respiratory intervals, because the highest number of detected heartbeat intervals or respiratory intervals at a position or distance indicates that the radar signal of the position or distance is appropriate to estimate heartbeat intervals or respiratory intervals.

A vital information acquisition apparatus employing an embodiment of the present invention may calculate tentative heartbeat intervals and/or respiratory intervals among plural positions or distances, and estimate heartbeat intervals or respiratory intervals using all or part of the tentative heartbeat intervals and/or respiratory intervals.

A vital information acquisition apparatus employing an embodiment of the present invention may evaluate the continuity of calculated heartbeat intervals, and eliminate calculated heartbeat intervals those have values largely different from nearby values, where the evaluation of the continuity includes standard deviation, average deviation, and median absolute deviation. A vital information acquisition apparatus employing an embodiment of the present invention may eliminate calculated heartbeat intervals, where the differences between those values and the average are more than three times the standard deviation.

A vital information acquisition apparatus employing an embodiment of the present invention may calculate the phase signals of the radar signals based on one of techniques using phase rotation information of the radar signals, where the techniques using phase rotation information of the radar signals include circle fitting technique. A circle fitting technique of a vital information acquisition apparatus employing an embodiment of the present invention is given by the minimization of the cost function D_{Z}:

$${D}_{Z} ( a , β , γ ) = {\sum }_{l = 1}^{L} {\vert {( {s}_{Il} - α )}^{2} + {( {s}_{Ql} - β )}^{2} - {γ}^{2} \vert}^{n} , \qquad ( 1 )$$

where α, β and γ are parameters for minimization, s_{Il} and s_{Ql} are the in-phase and quadrature signal components of the l-th data point, L is the data length in the time domain used for the calculation of the phase signals, and n is a positive number. A circle fitting technique of a vital information acquisition apparatus employing an embodiment of the present invention may employ the setting of n=2. A circle fitting technique of a vital information acquisition apparatus employing an embodiment of the present invention is given by the minimization of the cost function Du:

$${D}_{H} ( α , β ) = {\sum }_{l = 1}^{L} {\vert {d}_{l} - μ \vert}^{{n}_{H}} , \qquad ( 2 )$$

$$μ = \frac{1}{L} {\sum }_{l = 1}^{L} {d}_{l} , \qquad ( 3 )$$

$${d}_{l} = \sqrt{{( {s}_{I l} - α )}^{2} + {( {s}_{Ql} - β )}^{2}} , \qquad ( 4 )$$

where α and β are parameters for minimization of D_{H}. A circle fitting technique of a vital information acquisition apparatus employing an embodiment of the present invention may employ the setting of n_{H}=2. The phase signal of the l-th data point, s_{P}(l), is given by:

$${s}_{P} ( l ) = arg ( {s}_{Il} - {a}_{M} , {s}_{Q l} - {β}_{M} ) , \qquad ( 5 )$$

where α_{M} and β_{M} are the parameters after minimization of D_{Z} or D_{H}, and (a, b) represents a complex number with real part a and imaginary part b.

A vital information acquisition apparatus employing an embodiment of the 2.5 present invention may apply one of the normalization techniques to the radar signals in order to prevent data overflow. The radar signal of a vital information acquisition apparatus employing an embodiment of the present invention is given by:

$${s}_{{Il}^{′}} = {s}_{Il} / P , \qquad ( 6 )$$

$${s}_{{Ql}^{′}} = {s}_{Ql} / P , \qquad ( 7 )$$

$$P = \frac{1}{L} {\sum }_{l = 1}^{L} {\vert {s}_{Il}^{2} + {s}_{Ql}^{2} \vert}^{2} , \qquad ( 8 )$$

where s_{Il′} and s_{Ql}′ are the in-phase and quadrature signal components of the l-th data point after the application of one of the normalization techniques.

A vital information acquisition apparatus employing an embodiment of the present invention may calculate the initial value of the center of the phase rotation of the radar signals based on principal component analysis. An initial value of the center of the phase rotation of the radar signals employing an embodiment of the present invention is given by:

$$( {α}_{i} , {β}_{i} ) = {T}^{- 1} ⌈ ( 0 , P ) ] , \qquad ( 9 )$$

where αᵢ and βᵢ are the in-phase and quadrature components of the initial value of the center of the phase rotation of the radar signals for the minimization of Equation (1) or Equation (4), T⁻¹ is the inverse matrix of T, Tis the principal components analysis transformation matrix of the L×2 data matrix X, and the l-th row of X represents the in-phase and quadrature signal components of the l-th data point, S_{Il} and s_{Ql}. An initial value of the center of the phase rotation of the radar signals using a normalization technique employing an embodiment of the present invention is given by:

$$( {a}_{i}^{′} {β}_{i}^{′} ) = {T}^{- 1} [ ( 0 , 1 ) ] , \qquad ( 10 )$$

where α_{i′} and βᵢ′ are the in-phase and quadrature components of the initial value of the center of the phase rotation of the radar signals for the minimization using the normalization technique given by Equations (6), (7) and (8).

A vital information acquisition apparatus employing an embodiment of the present invention may calculate at least one phase-related function using a phase signal and the first- or higher-order derivatives of the phase signal, and estimate heartbeat intervals from the phase-related function using one of periodicity detection techniques; the periodicity detection techniques include autocorrelation, frequency analysis using Fourier transform, and zero-crossing detection algorithm.

A vital information acquisition apparatus employing an embodiment of the present invention may calculate at least one phase-related complex function using a phase signal and the first- or higher-order derivatives of the phase signal, and estimate heartbeat intervals from the phase-related function using one of periodicity detection techniques. A phase-related function employing an embodiment of the present invention, s_{PR}(t), is given by:

$${s}_{PR} ( t ) = {r}_{1} {s}_{P 1} ( t ) + {r}_{2} {s}_{P 2} ( t ) + {r}_{3} {s}_{P 3} ( t ) , \qquad ( 11 )$$

$${s}_{P 1} ( t ) = \{ \begin{matrix}1 & where {s}_{P}^{′} ( t ) ≧ 0 \\ - 1 & where {s}_{P}^{′} ( t ) < 0\end{matrix} , \qquad ( 12 )$$

$${s}_{P 2} ( t ) = \{ \begin{matrix}1 & where {s}_{P}^{″} ( t ) ≧ 0 \\ - 1 & where {s}_{P}^{″} ( t ) < 0\end{matrix} , \qquad ( 13 )$$

$${s}_{P 3} ( t ) = \{ \begin{matrix}1 & where {s}_{P}^{′′′} ( t ) ≧ 0 \\ - 1 & where {s}_{P}^{′′′} ( t ) < 0\end{matrix} , \qquad ( 14 )$$

where r₁, r₂, and r₃ are complex constants, S_{P}′(t), S_{P}″(t), and s_{P}′″(t) are the first-, second- and third-order derivatives of the phase signal, and f is the measurement time, that is the slow time. A phase-related function employing an embodiment of the present invention may employ a real constant, a pure imaginary constant, and a pure imaginary constant for r₁, r₂, and r₃, respectively.

A vital information acquisition apparatus employing an embodiment of the present invention may select head position for the estimation of heartbeat intervals, and select thorax or abdomen position for the estimation of respiratory intervals.

A vital information acquisition apparatus employing an embodiment of the present invention may use the transmitting antenna and/or the receiving antenna further employs narrow radar beams. The employment of narrow radar beams enables to limit scattering region, suppressing the measurement error in estimating heartbeat intervals caused by the variation of scattering region.

A vital information acquisition apparatus employing an embodiment of the present invention may use narrow radar beam, where the radar beam width is 20 cm or less. Pulse wave velocity is from 5 to 15 m/s, and thus this setting is supposed to suppress the measurement error in estimating heartbeat intervals caused by the variation of scattering region to 40 ms or less.

A vital information acquisition apparatus employing an embodiment of the present invention may employ frequency domain interferometry in order to detect the timing of heart ejection. A vital information acquisition apparatus employing an embodiment of the present invention may comprise an ultra-wideband millimeter-wave radar system which includes at least one transmitting antenna and at least one receiving antenna and is configured to transmit a plurality of ultra-wideband millimeter-waves to a subject and receive a plurality of ultra-wideband millimeter-waves reflected by the subject; and a controller comprising circuitry configured to convert a plurality of received ultra-wideband millimeter-waves to a plurality of radar signals, store the radar signals, calculate the phase signals of the radar signals, and calculate the first- or higher-order derivatives of each phase signal, and estimate heartbeat intervals and/or respiratory intervals from the phase signals and/or their derivatives using frequency domain interferometry.

A vital information acquisition apparatus employing an embodiment of the present invention may employ plural reference signals, and estimate heartbeat intervals, respiratory intervals and/or other biological information; other biological information includes cardiac output and blood pressure.

A vital information acquisition apparatus employing an embodiment of the present invention transmits microwaves to a subject and heartbeat intervals and respiratory intervals of the subject from the phase signals calculated using the plural radar signals. A vital information acquisition apparatus of the present invention according to an embodiment comprises a microwave radar system which includes at least one transmitting antenna and at least one receiving antenna and is configured to transmit a plurality of microwaves to a subject and receive a plurality of microwaves reflected by the subject; and a controller comprising circuitry configured to convert a plurality of received microwaves to a plurality of radar signals, store the radar signals, select plural positions or distances from the region of interest, calculate at least one phase signal from the radar signals at the selected positions or distances, and estimate heartbeat intervals and/or respiratory intervals from the phase signals using one of periodicity detection techniques: the periodicity detection techniques include autocorrelation, frequency analysis using Fourier transform, and zero-crossing detection algorithm.

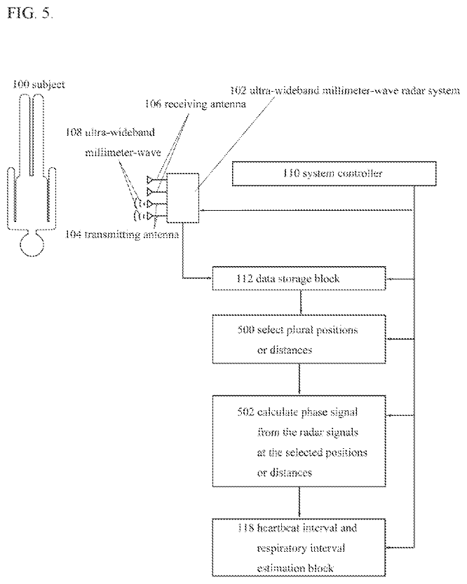

A vital information acquisition apparatus employing an embodiment of the present invention can employ an ultra-wideband millimeter-wave radar system. FIG. 5 shows a schematic diagram of a vital information acquisition apparatus employing an embodiment of the present invention. An ultra-wideband millimeter-wave radar system includes at least one transmitting antenna 104 and at least one receiving antenna 106. A plurality of ultra-wideband millimeter-waves 108 are transmitted to a subject 100. Transmitted ultra-wideband millimeter-waves can be modulated using one of pulse compression techniques, e.g., m-sequence. Transmitted ultra-wideband millimeter-waves 108 are reflected at the body surface of the subject 100. A plurality of ultra-wideband millimeter-waves reflected by the subject are received by receiving antennas 106. A system controller 110 comprising circuitry is configured to convert a plurality of received ultra-wideband millimeter-waves to a plurality of radar signals, store the radar signals 112, select plural positions or distances from the region of interest 500, calculate at least one phase signal from the radar signals at the selected positions or distances 502, and estimate heartbeat intervals and/or respiratory intervals from the phase signals and/or their derivatives using one of periodicity detection techniques 118. The periodicity detection techniques include autocorrelation, frequency analysis using Fourier transform, and zero-crossing detection algorithm.

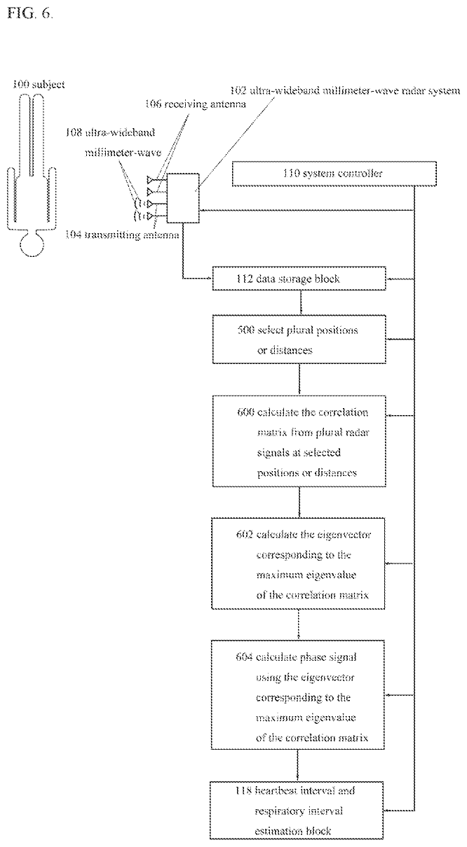

A vital information acquisition apparatus employing an embodiment of the present invention may calculate the phase signal using one of techniques based on principal component analysis, where the techniques based on principal component analysis include the calculation of the first principal component of the selected radar signals based on eigenvalue decomposition, iterative computation, and non-linear iterative partial least squares method. A calculation of the phase signal, s_{P}(l), using the first principal component of the selected radar signals based on eigenvalue decomposition employing an embodiment of the present invention is given by

$${s}_{P} ( l ) = {u ( l )}^{T} {v}_{max} , \qquad ( 19 )$$

$$u ( l ) = {[ {u}_{1} ( l ) , {u}_{2} ( l ) , … , {u}_{M} ( l ) ]}^{T} , \qquad ( 20 )$$

$${u}_{m} ( l ) = {s}_{I m} ( l ) + j {s}_{Q m} ( l ) , \qquad ( 21 )$$

where uₘ(l) is the radar signal at the m-th selected position or distance at the l-th data point, s_{Im}(l) and s_{Qm}(l) are the in-phase and quadrature signal components at the l-th data points, j is the imaginary unit, [ ]^{T} is the transpose of the matrix of [ ], and vₘₐₓ is the eigenvector corresponding to the maximum eigenvalue of the correlation matrix of u(l). A calculation of the correlation matrix of u(l), R, employing an embodiment of the present invention is given by

$$R = {\sum }_{l = 1}^{L} u ( l ) {u ( l )}^{H} , \qquad ( 22 )$$

where [ ]^{H} is the Hermitian transpose of the matrix of [ ]. FIG. 6 shows a schematic diagram of a vital information acquisition apparatus employing an embodiment of the present invention. An ultra-wideband millimeter-wave radar system includes at least one transmitting antenna 104 and at least one receiving antenna 106. A plurality of ultra-wideband millimeter-waves 108 are transmitted to a subject 100. Transmitted ultra-wideband millimeter-waves can be modulated using one of pulse compression techniques, e.g., m-sequence. Transmitted ultra-wideband millimeter-waves 108 are reflected at the body surface of the subject 100. A plurality of ultra-wideband millimeter-waves reflected by the subject are received by receiving antennas 106. A system controller 110 comprising circuitry is configured to convert a plurality of received ultra-wideband millimeter-waves to a plurality of radar signals, store the radar signals 112, select plural positions or distances from the region of interest 500, calculate the correlation matrix from plural radar signals at selected positions or distances 600, calculate the eigenvector corresponding to the maximum eigenvalue of the correlation matrix 602, calculate phase signal using the eigenvector corresponding to the maximum eigenvalue of the correlation matrix 604, and estimate heartbeat intervals and/or respiratory intervals from the phase signals and/or their derivatives using one of periodicity detection techniques 118.

The technique based on principal component analysis employing an embodiment of the present invention may employ one of expectation operators. The employment of expectation operator employing an embodiment of the present invention is given by the employment of the correlation matrix with weighted summation, R′, as the substitute for the correlation matrix with simple summation, R. Weighted summations include the employment of Hann window, Hamming window, and Gaussian window.

A vital information acquisition apparatus employing an embodiment of the present invention may select plural positions or distances with high signal intensity from the region of interest.

A vital information acquisition apparatus employing an embodiment of the present invention may apply at least one band-pass filter or high-pass filter to radar signals in order to extract the physiological signal of a subject.

A vital information acquisition apparatus employing an embodiment of the present invention may employ a band-pass filter or high-pass filter passes frequency components higher than 0.5 Hz for the estimation of heartbeat interval, because this setting can suppress most of the respiratory components and body movements and extract the physiological signal corresponding to heartbeat selectively.

A vital information acquisition apparatus employing an embodiment of the present invention may apply at least one band-pass filter or high-pass filter to phase signals in order to extract the physiological signal of a subject.

A vital information acquisition apparatus employing an embodiment of the present invention may further calculate the first- or higher-order derivatives of each phase signal, and estimate heartbeat intervals and/or respiratory intervals from the phase signals and/or their derivatives using one of periodicity detection techniques.

A vital information acquisition apparatus employing an embodiment of the present invention may calculate the first-, second- and third-order derivatives of each phase signal, and estimate heartbeat intervals and/or respiratory intervals from the phase signals and their first-, second- and third-order derivatives using one of periodicity detection techniques.

A vital information acquisition apparatus employing an embodiment of the present invention may apply one of multi-valued filters to the first-, second- and third-order derivatives of the phase signals, and configured to estimate heartbeat intervals and/or respiratory intervals from the phase signals and their filtered first-, second- and third-order derivatives using one of periodicity detection techniques; the multi-valued filters include binarization filter.

A vital information acquisition apparatus of the present invention according to an embodiment transmits microwaves to a subject and heartbeat intervals and respiratory intervals of the subject from the phase signals calculated using the plural radar signals. A vital information acquisition apparatus of the present invention according to an embodiment comprises a microwave radar system which includes at least one transmitting antenna and at least one receiving antenna and is configured to transmit a plurality of microwaves to a subject and receive a plurality of microwaves reflected by the subject: a microphone configured to receive a plurality of sound; and a controller comprising circuitry configured to convert a plurality of received microwaves to a plurality of radar signals, store the radar signals, select plural positions or distances from the region of interest, calculate at least one phase signal from the radar signals at the selected positions or distances, convert a plurality of sound to a plurality of sound signals, store the sound signals, process the sound signals such that an impact comprising sound pressure is calculated from the sound signals, and estimate heartbeat intervals, respiratory intervals, sleep stages, sleep apnea and/or hypopnea from the phase signals using one of periodicity detection techniques and/or the periodicity of the existence time distribution of the impact comprising sound pressure; the periodicity detection techniques include autocorrelation, frequency analysis using Fourier transform, and zero-crossing detection algorithm.

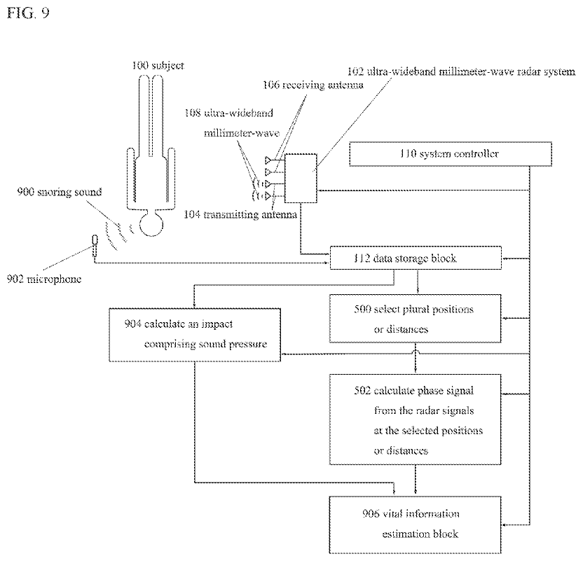

A vital information acquisition apparatus employing an embodiment of the present invention can employ an ultra-wideband millimeter-wave radar system. FIG. 9 shows a schematic diagram of a vital information acquisition apparatus employing an embodiment of the present invention. An ultra-wideband millimeter-wave radar system includes at least one transmitting antenna 104 and at least one receiving antenna 106. A plurality of ultra-wideband millimeter-waves 108 are transmitted to a subject 100. Transmitted ultra-wideband millimeter-waves can be modulated using one of pulse compression techniques. e.g., m-sequence. Transmitted ultra-wideband millimeter-waves 108 are reflected at the body surface of the subject 100. A plurality of ultra-wideband millimeter-waves reflected by the subject are received by receiving antennas 106. A plurality of sound including snoring sounds 900 are received by a microphone 902. A system controller 110 comprising circuitry is configured to convert a plurality of received ultra-wideband millimeter-waves to a plurality of radar signals, store the radar signals 112, select plural positions or distances from the region of interest 500, calculate at least one phase signal from the radar signals at the selected positions or distances 502, convert a plurality of sound to a plurality of sound signals, store the sound signals 112, process the sound signals such that an impact comprising sound pressure is calculated from the sound signals 904, and estimate heartbeat intervals, respiratory intervals, sleep stages, sleep apnea and/or hypopnea from the phase signals using one of periodicity detection techniques and/or the periodicity of the existence time distribution of the impact comprising sound pressure 906. The periodicity detection techniques include autocorrelation, frequency analysis using Fourier transform, and zero-crossing detection algorithm.

A vital information acquisition apparatus employing an embodiment of the present invention may detect snoring sound based on the periodicity of the existence time distribution of the impact comprising sound pressure.

A vital information acquisition apparatus employing an embodiment of the present invention may detect snoring sound based on the periodicity of the existence time distribution of the impact comprising sound pressure and the respiratory intervals estimated from the phase signals of the radar signals using one of periodicity detection techniques, because the respiratory interval estimated from the radar signals should be consistent with the existence time interval of the snoring sound.

A vital information acquisition apparatus employing an embodiment of the present invention may estimate sleep stages from the existence time distribution of snores, because the subject should be sleeping when he/she snores.

A vital information acquisition apparatus employing an embodiment of the present invention may estimate sleep apnea and hypopnea from the existence time distribution of snores and phase signals of the radar signals, because sleep apnea or hypopnea occurs when the subject sleeps, and the amplitude of the phase signal of the radar signals decreases when the movement of thorax or abdomen decreases.

A vital information acquisition method of the present invention according to an embodiment stores the radar signals, calculates the phase signals of the radar signals, and calculates the first- or higher-order derivatives of each phase signal, and estimates heartbeat intervals and respiratory intervals from the phase signals and/or their derivatives using one of periodicity detection techniques; the periodicity finding techniques include autocorrelation, frequency analysis using Fourier transform, and zero-crossing detection algorithm.

A vital information acquisition method employing an embodiment of the present invention stores the radar signals, selects plural positions or distances from the region of interest, calculates at least one phase signal from the radar signals at the selected positions or distances, and estimates heartbeat intervals and/or respiratory intervals from the phase signals using one of periodicity detection techniques; the periodicity detection techniques include autocorrelation, frequency analysis using Fourier transform, and zero-crossing detection algorithm.

A vital information acquisition method employing an embodiment of the present invention stores the radar signals, selects plural positions or distances from the region of interest, calculates at least one phase signal from the radar signals at the selected positions or distances, converts a plurality of sound to a plurality of sound signals, stores the sound signals, processes the sound signals such that an impact comprising sound pressure is calculated from the sound signals, and estimates heartbeat intervals, respiratory intervals, sleep stages, sleep apnea and/or hypopnea from the phase signals using one of periodicity detection techniques and sound signals and/or the periodicity of the existence time distribution of the impact comprising sound pressure; the periodicity detection techniques include autocorrelation, frequency analysis using Fourier transform, and zero-crossing detection algorithm.

### First Exemplary Embodiment

FIG. 1 shows a schematic diagram of a vital information acquisition apparatus employing an embodiment of the present invention. An ultra-wideband millimeter-wave radar system includes at least one transmitting antenna 104 and at least one receiving antenna 106. A plurality of ultra-wideband millimeter-waves 108 are transmitted to a subject 100. Transmitted ultra-wideband millimeter-waves can be modulated using one of pulse compression techniques, e.g. m-sequence. Transmitted ultra-wideband millimeter-waves 108 are reflected at the body surface of the subject 100. A plurality of ultra-wideband millimeter-waves reflected by the subject are received by receiving antennas 106. A system controller 110 comprising circuitry is configured to convert a plurality of received ultra-wideband millimeter-waves to a plurality of radar signals, store the radar signals 112, calculate the phase signals of the radar signals 114, and calculate the first- or higher-order derivatives of each phase signal 116, apply one of multi-valued filters to the first-, second- and third-order derivatives of the phase signals 200, and estimate heartbeat intervals from the phase signals and/or their derivatives using one of periodicity detection techniques 118. The multi-valued filters include binarization filter. The periodicity finding techniques include autocorrelation, frequency analysis using Fourier transform, and zero-crossing detection algorithm.

### Second Exemplary Embodiment

FIG. 2 shows a schematic diagram of a vital information acquisition apparatus employing an embodiment of the present invention. An ultra-wideband millimeter-wave radar system includes at least one transmitting antenna 104 and at least one receiving antenna 106. A plurality of ultra-wideband millimeter-waves 108 are transmitted to a subject 100. Transmitted ultra-wideband millimeter-waves can be modulated using one of pulse compression techniques, e.g. m-sequence. Transmitted ultra-wideband millimeter-waves 108 are reflected at the body surface of the subject 100. A plurality of ultra-wideband millimeter-waves reflected by the subject are received by receiving antennas 106. A system controller 110 comprising circuitry is configured to convert a plurality of received ultra-wideband millimeter-waves to a plurality of radar signals, store the radar signals 112, calculate the phase signals of the radar signals 114, and calculate the first- or higher-order derivatives of each phase signal 116, apply band-path filter to the phase signals of the radar signals, the radar signals, and/or the derivatives of the phase signals of the radar signals 300, apply one of multi-valued filters to the first-, second- and third-order derivatives of the phase signals after band-path filter application 200, and estimate heartbeat intervals from the phase signals after band-path application and/or their derivatives using one of periodicity detection techniques 118. The multi-valued filters include binarization filter. The periodicity finding techniques include autocorrelation, frequency analysis using Fourier transform, and zero-crossing detection algorithm.

### Third Exemplary Embodiment

FIG. 3 shows a schematic diagram of a vital information acquisition apparatus employing an embodiment of the present invention. An ultra-wideband millimeter-wave radar system includes at least one transmitting antenna 104 and at least one receiving antenna 106. A plurality of ultra-wideband millimeter-waves 108 are transmitted to a subject 100. Transmitted ultra-wideband millimeter-waves can be modulated using one of pulse compression techniques, e.g. m-sequence. Transmitted ultra-wideband millimeter-waves 108 are reflected at the body surface of the subject 100. A plurality of ultra-wideband millimeter-waves reflected by the subject are received by receiving antennas 106. A system controller 110 comprising circuitry is configured to convert a plurality of received ultra-wideband millimeter-waves to a plurality of radar signals, store the radar signals 112, calculate the phase signals of the radar signals 114, select appropriate position or distance from the region of interest for the estimation of respiratory intervals 300, calculate the first- or higher-order derivatives of each phase signal 116, apply one of multi-valued filters to the first-, second- and third-order derivatives of the phase signals 200, select appropriate position or distance from the region of interest for the estimation of heartbeat intervals 302, and estimate heartbeat intervals 306 and/or respiratory intervals 304 from the phase signals and/or their derivatives using one of periodicity detection techniques, where the appropriate position or distance for the estimation of heartbeat intervals and the appropriate position or distance for the estimation of respiratory intervals do not have to be the same. The selection of appropriate position for respiratory interval estimation may follow the calculation of the derivatives of phase signals.

### Forth Exemplary Embodiment

FIG. 4 shows a schematic diagram of a vital information acquisition apparatus employing an embodiment of the present invention. An ultra-wideband millimeter-wave radar system includes at least one transmitting antenna 104 and at least one receiving antenna 106. A plurality of ultra-wideband millimeter-waves 108 are transmitted to a subject 100. Transmitted ultra-wideband millimeter-waves can be modulated using one of pulse compression techniques, e.g. m-sequence. Transmitted ultra-wideband millimeter-waves 108 are reflected at the body surface of the subject 100. A plurality of ultra-wideband millimeter-waves reflected by the subject are received by receiving antennas 106. A system controller 110 comprising circuitry is configured to convert a plurality of received ultra-wideband millimeter-waves to a plurality of radar signals, store the radar signals 112, calculate the phase signals of the radar signals 114, and calculate the first- or higher-order derivatives of each phase signal 116, apply band-path filter to the phase signals of the radar signals and/or the derivatives of the phase signals of the radar signals 400, apply one of multi-valued filters to the first-, second- and third-order derivatives of the phase signals after band-path filter application 200, and estimate heartbeat intervals and/or respiratory intervals from the phase signals after band-path application and/or their derivatives using one of periodicity detection techniques 118.

### Fifth Exemplary Embodiment

A vital information acquisition apparatus employing an embodiment of the present invention may employ the following configuration. An ultra-wideband millimeter-wave radar system includes at least one transmitting antenna and at least one receiving antenna. A plurality of ultra-wideband millimeter-waves are transmitted to a subject. Transmitted ultra-wideband millimeter-waves can be modulated using one of pulse compression techniques, e.g. m-sequence. Transmitted ultra-wideband millimeter-waves are reflected at the body surface of the subject. A plurality of ultra-wideband millimeter-waves reflected by the subject are received by receiving antennas. A system controller comprising circuitry is configured to convert a plurality of received ultra-wideband millimeter-waves to a plurality of radar signals, store the radar signals, calculate the phase signals of the radar signals, select appropriate position or distance from the region of interest for the estimation of respiratory intervals, estimate respiratory intervals from the phase signals and/or their derivatives of the appropriate position or distance for respiratory interval estimation using one of periodicity detection techniques, calculate the first- or higher-order derivatives of each phase signal, apply one of multi-valued filters to the first-, second- and third-order derivatives of the phase signals, select appropriate position or distance from the region of interest for the estimation of heartbeat intervals, and estimate heartbeat intervals from the phase signals and/or their derivatives of the appropriate position or distance for heartbeat interval estimation using one of periodicity detection techniques. The vital information acquisition apparatus employing an embodiment of the present invention may calculate the phase signals of the radar signals by the minimization of the cost function Du given by

$${D}_{H} ( {α}^{′} , {β}^{′} ) = {\sum }_{l = 1}^{L} {\vert {d}_{l}^{′} - {μ}^{′} \vert}^{{n}_{H}} , \qquad ( 15 )$$

$${μ}^{′} = \frac{1}{L} {\sum }_{l = 1}^{L} {d}_{l}^{′} , \qquad ( 16 )$$

$${d}_{l}^{′} = \sqrt{{( {s}_{Il}^{′} - {α}^{′} )}^{2} + {( {s}_{Ql}^{′} - {β}^{′} )}^{2}} , \qquad ( 17 )$$

where α′ and β′ are parameters for minimization of D_{H}′, and the initial values of α′ and β′ are given by Equation (10). The phase-related function employing an embodiment of the present invention given by Equation (11) may employ the parameters of r₁, r₂, and r₃ given by:

$$( {r}_{1} , {r}_{2} , {r}_{3} ) = ( {a}_{1} , {a}_{2} j , {a}_{3} j ) , \qquad ( 18 )$$

where a₁, a₂, and a₃ are real numbers, j is the imaginary unit. A vital information acquisition apparatus employing an embodiment of the present invention may employ positive numbers for a₁, a₂, and a₃, including the settings of (a₁, a₂, and a₃)=(1, 1, 0.3), (1, 1, 0.4), (1, 1, 0.5), (1, 1, 0.6), (1, 1, 0.7), (1, 1, 0.8), (1, 1, 0.9), (1, 1, 1), (1, 0.3, 1), (1, 0.4, 1), (1, 0.5, 1), (1, 0.6, 1), (1, 0.7, 1), (1, 0.8, 1), (1, 0.9, 1), (1, 0.3, 0.3). (1, 0.4, 0.4), (1, 0.5, 0.5), (1, 0.6, 0.6), (1, 0.7, 0.7), (1, 0.8, 0.8), and (1, 0.9, 0.9).

### Sixth Exemplary Embodiment

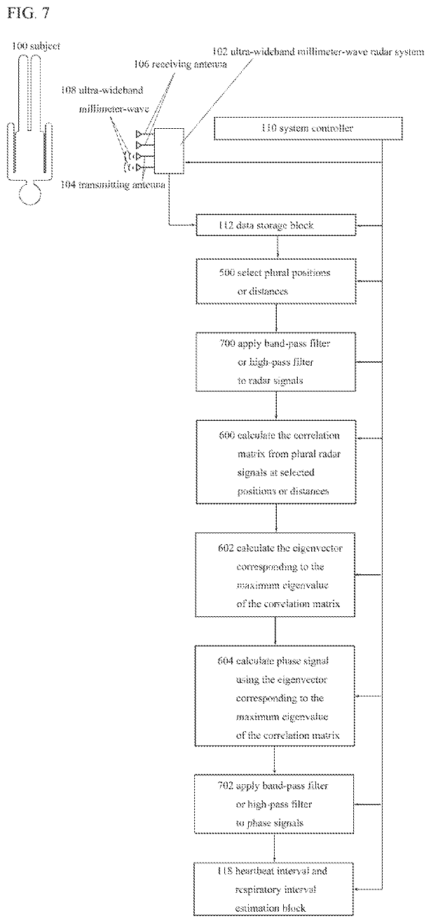

FIG. 7 shows a schematic diagram of a vital information acquisition apparatus employing an embodiment of the present invention. An ultra-wideband millimeter-wave radar system includes at least one transmitting antenna 104 and at least one receiving antenna 106. A plurality of ultra-wideband millimeter-waves 108 are transmitted to a subject 100. Transmitted ultra-wideband millimeter-waves can be modulated using one of pulse compression techniques, e.g., m-sequence. Transmitted ultra-wideband millimeter-waves 108 are reflected at the body surface of the subject 100. A plurality of ultra-wideband millimeter-waves reflected by the subject are received by receiving antennas 106. A system controller 110 comprising circuitry is configured to convert a plurality of received ultra-wideband millimeter-waves to a plurality of radar signals, store the radar signals 112, select plural positions or distances with high signal intensity from the region of interest 500, apply band-pass filter or high-pass filter to radar signals 700, calculate the correlation matrix from plural radar signals at selected positions or distances 600, calculate the eigenvector corresponding to the maximum eigenvalue of the correlation matrix 602, calculate phase signal using the eigenvector corresponding to the maximum eigenvalue of the correlation matrix 604, apply band-pass filter or high-pass filter to phase signals 702, and estimate heartbeat intervals and/or respiratory intervals from the phase signals and/or their derivatives using one of periodicity detection techniques 118. Application of band-pass filter or high-pass filter to radar signals may be followed by the selection of plural positions or distances.

### Seventh Exemplary Embodiment

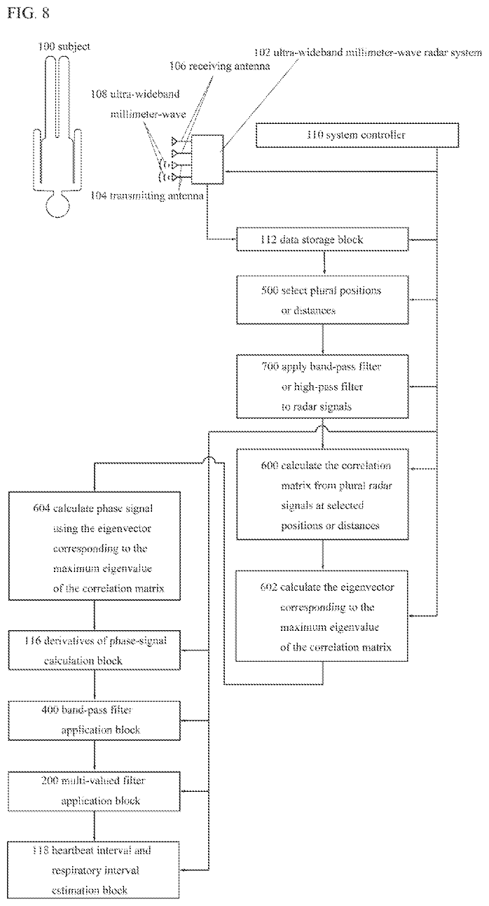

FIG. 8 shows a schematic diagram of a vital information acquisition apparatus employing an embodiment of the present invention. An ultra-wideband millimeter-wave radar system includes at least one transmitting antenna 104 and at least one receiving antenna 106. A plurality of ultra-wideband millimeter-waves 108 are transmitted to a subject 100. Transmitted ultra-wideband millimeter-waves can be modulated using one of pulse compression techniques, e.g., m-sequence. Transmitted ultra-wideband millimeter-waves 108 are reflected at the body surface of the subject 100. A plurality of ultra-wideband millimeter-waves reflected by the subject are received by receiving antennas 106. A system controller 110 comprising circuitry is configured to convert a plurality of received ultra-wideband millimeter-waves to a plurality of radar signals, store the radar signals 112, select plural positions or distances with high signal intensity from the region of interest 500, apply band-pass filter or high-pass filter to radar signals 700, calculate the correlation matrix from plural radar signals at selected positions or distances 600, calculate the eigenvector corresponding to the maximum eigenvalue of the correlation matrix 602, calculate phase signal using the eigenvector corresponding to the maximum eigenvalue of the correlation matrix 604, and calculate the first- or higher-order derivatives of each phase signal 116, apply band-pass filter to the phase signals of the radar signals, the radar signals, and/or the derivatives of the phase signals of the radar signals 400, apply one of multi-valued filters to the first-, second- and third-order derivatives of the phase signals after band-pass filter application 200, and estimate heartbeat intervals from the phase signals after band-pass application and/or their derivatives using one of periodicity detection techniques 118. The multi-valued filters include binarization filter. The periodicity detection techniques include autocorrelation, frequency analysis using Fourier transform, and zero-crossing detection algorithm.

### Eighth Exemplary Embodiment

FIG. 9 shows a schematic diagram of a vital information acquisition apparatus employing an embodiment of the present invention. An ultra-wideband millimeter-wave radar system includes at least one transmitting antenna 104 and at least one receiving antenna 106. A plurality of ultra-wideband millimeter-waves 108 are transmitted to a subject 100. Transmitted ultra-wideband millimeter-waves can be modulated using one of pulse compression techniques, e.g., m-sequence. Transmitted ultra-wideband millimeter-waves 108 are reflected at the body surface of the subject 100. A plurality of ultra-wideband millimeter-waves reflected by the subject are received by receiving antennas 106. A plurality of sound including snoring sounds 900 are received by a microphone 902. A system controller 110 comprising circuitry is configured to convert a plurality of received ultra-wideband millimeter-waves to a plurality of radar signals, store the radar signals 112, select plural positions or distances from the region of interest 500, calculate at least one phase signal from the radar signals at the selected positions or distances 502, convert a plurality of sound to a plurality of sound signals, store the sound signals 112, process the sound signals such that an impact comprising sound pressure is calculated from the sound signals 904, and estimate heartbeat intervals, respiratory intervals, sleep stages, sleep apnea and/or hypopnea from the phase signals using one of periodicity detection techniques and/or the periodicity of the existence time distribution of the impact comprising sound pressure 906. The periodicity detection techniques include autocorrelation, frequency analysis using Fourier transform, and zero-crossing detection algorithm.

### REFERENCE SIGNS LIST

100 subject
102 ultra-wideband millimeter-wave radar system
104 transmitting antenna
106 receiving antenna
108 ultra-wideband millimeter-wave
110 system controller
112 data storage block
114 phase signal calculation block
116 derivatives of phase-signal calculation block
118 heartbeat interval and respiratory interval estimation block
200 multi-valued filter application block
300 select appropriate position for respiratory interval estimation
302 select appropriate position for heartbeat interval estimation
304 respiratory interval estimation block
306 heartbeat interval estimation block
400 band-path filter application block
500 select plural positions or distances
502 calculate phase signal from the radar signals at the selected positions or distances
600 calculate the correlation matrix from plural radar signals at selected positions or distances
602 calculate the eigenvector corresponding to the maximum eigenvalue of the correlation matrix
604 calculate phase signal using the eigenvector corresponding to the maximum eigenvalue of the correlation matrix
700 apply band-pass filter or high-pass filter to radar signals
702 apply band-pass filter or high-pass filter to phase signals
900 snoring sound
902 microphone
904 calculate an impact comprising sound pressure
906 vital information estimation block

1. A vital information acquisition apparatus comprising:
a microwave radar system configured to:
transmit a plurality of microwaves to a subject and
receive a plurality of microwaves reflected by the subject; and
a controller comprising circuitry configured to:
convert the received plurality of microwaves to a plurality of radar signals,
obtain a plurality of phase signals by calculating each of the plurality of phase signals of each of the plurality of radar signals,
calculate one or more derivatives of the each phase signal, and
estimate biological information including heartbeat intervals and/or respiratory' intervals from the phase signals and the derivatives.

2. The vital information acquisition apparatus according to claim 1, wherein the estimating of the heartbeat intervals and/or respiratory' intervals includes using a periodicity detection technique.

3. The vital information acquisition apparatus according to claim 2, wherein the periodicity detection technique is selected from the group consisting of autocorrelation, frequency analysis using Fourier transform, and zero-crossing detection algorithm.

4. The vital information acquisition apparatus according to claim 3, wherein the estimating of the biological information including heartbeat intervals and/or respiratory' intervals includes applying a multi-valued filter.

5. The vital information acquisition apparatus according to claim 4, wherein the multi-valued filter includes a binarization filter.

6. The vital information acquisition apparatus according to claim 5, wherein the circuitry is further configured to select appropriate position or distance from the region of interest in order to estimate respiratory intervals, and estimate respiratory intervals from the phase signals and/or their derivatives of the appropriate position or distance.

7. The vital information acquisition apparatus according to claim 6, wherein the appropriate position or the appropriate distance for the estimation of heartbeat intervals is different from the appropriate position or distance for the estimation of respiratory intervals.

8. (canceled)

9. (canceled)

10. (canceled)

11. The vital information acquisition apparatus according to claim 1, wherein the calculating of the phase signals of the radar signals is based on a technique using phase rotation information of the radar signals.

12. (canceled)

13. (canceled)

14. (canceled)

15. (canceled)

16. (canceled)

17. (canceled)

18. (canceled)

19. (canceled)

20. The vital information acquisition apparatus according to claim 1, wherein the circuitry is further configured to employ plural reference signals, and wherein the biological information further includes cardiac output and blood pressure based on the plural reference signals.

21. A vital information acquisition apparatus comprising:
a microwave radar system configured to:
transmit a plurality of microwaves to a subject and
receive a plurality of microwaves reflected by the subject; and
a controller comprising circuitry configured to:
convert a plurality of received microwaves to a plurality of radar signals,
select plural positions or distances from a region of interest,
obtain a plurality of phase signals by calculating each of the plurality of phase signals of each of the plurality of radar signals at the selected positions or distances,
calculate one or more derivatives of the each phase signal, and
estimate biological information including heartbeat intervals and/or respiratory intervals from the phase signals and the derivatives.

22. A vital information acquisition apparatus comprising:
an ultra-wideband millimeter-wave radar system is configured to
transmit a plurality of ultra-wideband millimeter-waves to a subject and
receive a plurality of ultra-wideband millimeter-waves reflected by the subject; and
a controller comprising circuitry configured to:
convert the received plurality of ultra-wideband millimeter-waves to a plurality of radar signals,
select plural positions or distances from a region of interest,
obtain a plurality of phase signals by calculating each of the plurality of phase signals of each of the plurality of radar signals at the selected plural positions or distances,
calculate one or more derivatives of the each phase signal, and
estimate biological information including heartbeat intervals and/or respiratory intervals from the phase signals and the derivatives.

23. The vital information acquisition apparatus according to claim 22, wherein the calculating of the phase signals uses a technique based on a principal component analysis.

24. The vital information acquisition apparatus according to claim 23, wherein the technique based on the principal component analysis is selected from the group consisting of the calculation of the first principal component of the selected radar signals based on eigenvalue decomposition, iterative computation, and non-linear iterative partial least squares method.

25. The vital information acquisition apparatus according to claim 22, wherein the selecting of the plural positions or distances from the region of interest includes selecting of the plural positions or distances with a high signal intensity from the region of interest.

26. The vital information acquisition apparatus according to claim 22, wherein the circuitry further configured to apply at least one of a band-pass filter and a high-pass filter to the plurality of radar signals, to obtain the plurality of phase signals.

27. (canceled)

28. (canceled)

29. (canceled)

30. (canceled)

31. (canceled)

32. A vital information acquisition apparatus comprising:
a microwave radar system configured to
transmit a plurality of microwaves to a subject and
receive a plurality of microwaves reflected by the subject;
a microphone configured to receive a plurality of sounds; and
a controller comprising circuitry configured to:
convert the received plurality of microwaves to a plurality of radar signals,
select plural positions or distances from a region of interest,
obtain a plurality of phase signals by calculating each of the plurality of phase signals of each of the plurality of radar signals at the selected plural positions or distances,
calculate one or more derivatives of the each phase signal,
convert the plurality of sounds to a plurality of sound signals,
process the plurality of sound signals to calculate an impact comprising sound pressure from the plurality of sound signals, and
estimate biological information including heartbeat intervals, respiratory intervals, sleep stages, sleep apnea and/or hypopnea from the phase signals and the derivatives, using either one or both of a periodicity detection technique and a periodicity of an existence time distribution of the impact comprising sound pressure.

33. A vital information acquisition apparatus comprising:
an ultra-wideband millimeter-wave radar system configured to
transmit a plurality of ultra-wideband millimeter-waves to a subject and
receive a plurality of ultra-wideband millimeter-waves reflected by the subject;
a microphone configured to receive a plurality of sound; and
a controller comprising circuitry configured to:
convert the received plurality of ultra-wideband millimeter-waves to a plurality of radar signals,
select plural positions or distances from a region of interest,
obtain a plurality of phase signals by calculating each of the plurality of phase signals of each of the plurality of radar signals at the selected plural positions or distances,
calculate one or more derivatives of the each phase signal,
convert the plurality of sounds to a plurality of sound signals,
process the plurality of sound signals to calculate an impact comprising sound pressure from the plurality of sound signals, and
estimate biological information including heartbeat intervals, respiratory intervals, sleep stages, sleep apnea and/or hypopnea from the phase signals and the derivatives, using either one or both of a periodicity detection technique and a periodicity of the existence time distribution of the impact comprising sound pressure.

34. The vital information acquisition apparatus according to claim 33, wherein the circuitry is further configured to detect snoring sound based on the periodicity of the existence time distribution of the impact comprising sound pressure.

35. (canceled)

36. The vital information acquisition apparatus according to claim 34, wherein the circuitry is further configured to estimate sleep stages from the existence time distribution of snores.

37. The vital information acquisition apparatus according to claim 34, wherein the circuitry is further configured to estimate sleep apnea and hypopnea from tire existence time distribution of snores and the phase signals of tire radar signals.

38. (canceled)

39. (canceled)

40. (canceled)
